(19) United States
(12) Patent Application Publication
(10) Pub. No.: US 2026/0041849 A1
(43) Pub. Date: Feb. 12, 2026

(54) BLOW-FILL-SEAL (BFS) SYRINGE CARTRIDGE SYSTEMS AND METHODS

(71) Applicant: Koska Family Limited, East Sussex (GB)

(72) Inventors: Jay S. WALKER, Ridgefield, CT (US); March Andrew KOSKA, Dartmouth (GB); Harris RYDER, Redruth (GB); Max Kingsley HANNON, London (GB)

(21) Appl. No.: 19/363,438

(22) Filed: Oct. 20, 2025

Related U.S. Application Data
(63) Continuation of application No. PCT/US24/25736, filed on Apr. 22, 2024.
(60) Provisional application No. 63/461,214, filed on Apr. 21, 2023.
(60) Provisional application No. 63/463,879, filed on May 3, 2023.

(51) Int. Cl. A61M 5/24; A61M 5/31

(52) U.S. Cl. CPC A61M 5/2425; A61M 5/2459; A61M 2005/2437; A61M 2005/3125

(57) ABSTRACT

A syringe cartridge assembly includes a syringe housing, a compressible Blow-Fill-Seal (BFS) cartridge at least partially positionable within the syringe housing, and having a fluid agent. In at least some embodiments, the compressible cartridge includes a compressible bellows segment configured to transition between an initial condition and an at least partially compressed condition. A plunger is configured for movement within the syringe housing to cause transition of the compressible bellow segment from the initial condition to the at least partially compressed condition to and push the cartridge into a double-ended cannulated needle of a deliver assembly attached to the syringe housing, causing one end of the needle to penetrate the compressible BFS cartridge and dispense the fluid agent into a patient via the opposing end thereof.

### CLAIM OF PRIORITY

The present application is a Continuation Application of PCT Application No. PCT/US24/25736, filed on Apr. 22, 2024 in the name of Koska et al. and titled BLOW-FILL-SEAL (BFS) SYRINGE CARTRIDGE SYSTEMS AND METHODS, which PCT Application claims priority to (i) U.S. Provisional Application No. 63/461,214, filed on Apr. 21, 2023 in the name of Koska et al. and titled SYRINGE CARTRIDGE SYSTEM; and (ii) U.S. Provisional Application No. 63/463,879, filed on May 3, 2023 in the name of Koska et al. and titled BLOW-FILL-SEAL (BFS) CARTRIDGE TRAY SYSTEM. The entirety of each of the foregoing Applications is incorporated by reference herein for all purposes.

### BACKGROUND

Staggering numbers of people become infected, severely ill and/or die from a variety of diseases each year, some of which are preventable (or the severity of which could have been mitigated) through injectable medicines, medicaments, vaccines, drug products or other fluid agents (collectively “injectable medicines” herein, injectable medicines referring to fluid agents delivered via use of a syringe or other needle-type medical delivery device). Although injectable medicines have led to a decline in the number of cases of several infectious diseases (or the severity of symptoms or instances of death resulting therefrom) and/or ability to better manage ongoing health conditions such as diabetes, and have also become useful for administration of other medicaments such as contraceptives, the need for additional innovation in injectable medicines remains because the demand for effective contraceptive programs and control of existing and emerging diseases continues to grow and existing solutions leave room for further improvements. Applicant has previously invented various methods and systems intended to address the growing demand for injectable medicines through single-dose, pre-filled blow-fill-seal (BFS) injector devices that comprise one or more benefits, such as (i) being manufacturable in a cost effective manner, in large quantities and at short notice to meet unanticipated peaks in demand; (ii) incorporating simplicity of design that lends itself to administration by users who have little or no medical training (e.g., are suitable for self-injection); and/or (iii) including features that minimize risk of re-use or otherwise prioritize safety concerns. Applicant continues to innovate in this field by developing various new and beneficial systems, devices and components that will provide additional options and opportunities for drug manufacturers to select a single-dose, pre-filled delivery system that best fits their needs.

### SUMMARY

Embodiments described herein are directed to further improvements in BFS injector devices, including syringe-style cartridge assemblies and associated methods of use in delivering a fluid agent (e.g., a vaccine, a drug, a medicament, etc.). Moreover, embodiments described herein are directed to a syringe cartridge assembly including a compressible cartridge (e.g., a BFS cartridge) which is positionable within a syringe-style or barrel-style housing (also referred to as “syringe housing”, for purposes of brevity) of the assembly. The compressible cartridge may be coupled to or placed within the syringe housing during manufacture or, alternatively, by a user handling the cartridge at the operative site. In the latter situation, the compressible cartridges containing the fluid agent may be stored independent of the components of the syringe housing in a container (e.g., a refrigeration container if needed for preservation purposes), and then assembled when needed to deliver one or more doses. In illustrative embodiments, the syringe cartridge assembly includes a plunger which is advanceable within the syringe housing to compress the compressible cartridge against one end of the needle to cause the needle to penetrate the compressible cartridge to access the fluid agent for delivery to the patient.

In accordance with some embodiments, the compressible cartridge and/or the syringe housing may include one or more automatic disabling features which minimize the potential and/or eliminate removal of the compressible cartridge from the syringe housing once appropriately positioned for use in delivering the fluid agent. Such a feature may minimizes cross-contamination amongst multiple users and addresses sterility concerns. In illustrative embodiments, one automatic disabling feature includes cooperating disabling structure on the compressible cartridge and within the interior of the syringe housing which engage when the compressible cartridge is positioned within the syringe housing (at an initial position or condition), and prior to activation of the plunger. In certain embodiments, the plunger includes grasper structure which couples with the compressible cartridge to assist in positioning the compressible cartridge within the syringe housing to assume the initial condition with the cooperating disabling structure engaged. Thereafter, any attempts to remove the compressible cartridge from the syringe housing via the plunger will be prevented or hindered by the novel design and characteristics of the cooperating disabling structure. More specifically, as described in detail hereinbelow, in illustrative embodiments, the force(s) required to overcome the disabling structure between the compressible cartridge and the syringe housing is greater than the force(s) holding the grasper structure of the plunger onto the compressible cartridge. Thus, any attempt to remove the compressible cartridge from the syringe housing through withdrawal of the plunger causes the grasper structure to release the compressible cartridge, with the compressible cartridge thus remaining in the initial condition within the syringe housing.

In some embodiments, the compressible cartridge comprises a bellows structure having a series of individual bellows or comprising a plurality of concertinaed portions, which compress when the compressible cartridge is transitioned from the initial condition to an activated or compressed position or condition via movement of the plunger. In the compressed condition, the bellows structure expands radially (in cross-sectional dimension or diameter) within the syringe housing. In illustrative embodiments, one or more of the individual bellows clears or engages an internal annular rib within the syringe housing when in this compressed condition. The features of the internal annular rib and the compressed bellows structure provide an additional and/or alternative disabling feature of the assembly preventing removal and reuse of the expended compressible cartridge (i.e., after moving to the activated condition). Similar to the disabling feature described hereinabove, the force(s) required to overcome the disabling structure between the compressed bellows structure and the internal annular rib of the syringe housing may be greater than the force(s) holding the grasper structure of the plunger onto the compressible cartridge. Thus, retraction of the plunger causes release of the grasper structure from the expended compressible cartridge. As a further advantage, the disabling feature ensures the bellows structure does not reopen which may otherwise cause blood or body fluids to be drawn into the cartridge under vacuum.

While embodiments described herein refer to a bellow-style cartridge, other geometric configurations of compressible BFS cartridges may be utilized, so long as the BFS cartridge is compressible via depression of the plunger. In various embodiments, a fluid agent is filled into the compressible BFS cartridge while the BFS cartridge is being manufactured, and the BFS cartridge is sealed during the manufacturing process in a manner that allows the fluid agent to remain sterile and unexposed to the air until it is eventually penetrated by a needle of the syringe assembly as described herein.

The compressible cartridge may be used with a number of syringe housings which may be specifically made for the compressible cartridge. It is also envisioned that existing molds used to manufacture current syringe housings may be readily modified to accommodate the compressible cartridge. Thus, the compressible cartridge may be supplied with or without a syringe housing.

In one illustrative embodiment, a syringe assembly comprises (i) a syringe housing; (ii) a compressible cartridge at least partially positionable within the syringe housing and having a fluid agent stored therein; and (iii) a plunger configured for movement within the syringe housing to cause transition of the compressible cartridge from an initial condition to an at least partially compressed condition (e.g., by compressing at least the compressible bellows segment of the compressible cartridge, in embodiments in which the compressible cartridge is of a bellows type configuration) and (iv) a delivery member mountable to the syringe housing and configured for penetrating the compressible cartridge during transition of the compressible cartridge to the at least partially compressed condition to deliver the fluid agent to a subject. In at least some embodiments, the delivery member comprises a cannula or cannulated needle, one end of which penetrates the compressible cartridge when the cartridge is pushed into it via the plunger and the other end of which penetrates the skin of a patient to deliver the fluid agent from the compressible cartridge into the patient.

In an embodiment, a compressible bellows segment of a compressible cartridge is configured to cooperatively engage internal structure of the syringe housing upon transition to the at least partially compressed condition thereby preventing removal of the compressible cartridge from the syringe housing and impeding transition toward the initial condition of the compressible cartridge. In some embodiments, the compressible bellows segment of the compressible cartridge is configured to expand during transition to the at least partially compressed condition of the compressible cartridge. In certain embodiments, the syringe housing includes an internal annular rib segment whereby the internal annular rib segment is the internal structure of the syringe housing. In other embodiments, the internal annular rib segment extends along a major portion of an inner dimension of the syringe housing. In one embodiment, the internal annular rib segment is configured to engage one or more folds or concertinaed and contracted portions of the compressible bellows segment when in the at least partially compressed condition of the compressible cartridge.

In some embodiments, the plunger comprises a grasper component configured to engage the compressible cartridge to secure the compressible cartridge relative to the plunger. In certain embodiments, the grasper component of the plunger includes multiple grasping legs configured to deflect outwardly to engage the compressible cartridge. In other embodiments, the multiple grasping legs of the grasper component are configured to engage a proximal collar of the compressible cartridge. In some embodiments, the grasper component is configured to cooperate with the proximal collar of the compressible cartridge wherein a force required to release the grasper component from the proximal collar is less than a force required to release the one or more folds of the compressible bellows segment from the internal annular rib segment of the syringe housing such that the grasper component releases the plunger in response to retracting movement of the plunger when in the at least partially compressed condition of the compressible cartridge.

In certain embodiments, the compressible cartridge includes a distal collar and wherein the syringe housing includes an internal annular shelf segment whereby the internal annular shelf segment cooperates with the distal collar of the compressible cartridge to secure the compressible cartridge within the syringe housing when in the initial condition of the compressible cartridge. In some embodiments, the grasper component is configured to cooperate with the proximal collar of the compressible cartridge wherein a force required to release the grasper component from the proximal collar is less than a force required to release the distal collar from the internal annular shelf segment of the syringe housing such that the grasper component releases the plunger in response to retracting movement of the plunger when in the initial condition of the compressible cartridge.

In some embodiments, the plunger comprises an anti-activating component having a first pre-activation condition preventing movement of the plunger and dispensing of the fluid agent and a second activated condition enabling movement of the plunger and dispensing of the fluid agent. In embodiments, the anti-activating component includes a flag segment depending outwardly relative to an axis of the plunger when in the first pre-activation condition and movable to the second activated condition to enable movement of the plunger. In certain embodiments, the flag segment is detachable relative to the plunger to assume the second activated condition. In other embodiments, the anti-activating component includes one or more deflectable arms configured to move from a radial outward condition corresponding to the first pre-activation condition and a radial inward condition corresponding to the second activated condition. In embodiments, the anti-activating component includes multiple deflectable arms. In other embodiments, the one or more deflectable arms are configured to be received within the syringe housing to enable movement of the plunger when in the radial inward condition.

In embodiments, the compressible cartridge includes visual indicia to facilitate identification of one of the fluid agent or type of compressible cartridge. Such indicia may include specific geometries, colors, labels or human or machine-readable codes. For example, in some embodiments different geometries on at least one portion of a compressible cartridge may indicate different length and/or gauge cannula of a delivery assembly that the cartridge is supposed to be used with.

In other embodiments, the delivery member defines a subject end configured for application to the subject and an opposed cartridge end configured for penetrating the compressible cartridge. In some embodiments, the cartridge end of the delivery member includes piercing structure configured for piercing the compressible cartridge.

In another illustrative embodiment, a medical tray apparatus comprises a base tray defining one or more cartridge receptacles for holding one or more cartridge and a cover positionable over the base tray. At least one of the base tray and the cover includes one of an insulative material or a cooling medium. In embodiments, the base tray defines a plurality of individual cartridge receptacles with each individual cartridge receptacle accommodating an individual cartridge containing a fluid agent. In other embodiments, each individual cartridge receptacle includes a mount configured to selectively engage the individual cartridge. In some embodiments, a plurality of individual cartridges are at least partially disposed within respective individual cartridge receptacles of the base tray with each individual cartridge having a fluid agent therein. In certain embodiments, a seal is positionable over the base tray beneath the cover. In embodiments, the seal includes perforations to permit selective removal of portions of the seal to enable access to particular ones of the individual cartridges. In other embodiments, the seal includes a plurality of perforated lines.

In certain embodiments, one or more additional base trays may be mountable to the first-mentioned base tray in stacked relation. In other embodiments, one or more second base trays and/or a second cover is mountable to the first-mentioned base tray and the first-mentioned cover in stacked relation.

In some embodiments, the base tray includes at least first and second tray segments. The first and second tray segments may be detachable relative to each other, allowing for a more modular configuration. In embodiments, the first tray segment defines a first row of individual cartridge receptacles and the second tray segment defines a second row of individual cartridge receptacles, and be manufactured such that a user may readily detach one row of the tray from the others in order to use the cartridges stored therein.

In embodiments, at least one of the base tray and the cover comprises insulative material.

In other embodiments, the at least one of the base tray and the cover comprises the cooling medium. In certain embodiments, the cooling medium is embodied within the at least one of the base tray and the cover. In other embodiments, the cooling medium is at least partially enclosed within multiple layers of the at least one of the base tray and the cover.

Any of the various innovations and embodiments included in the disclosed subject matter herein can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

An understanding of embodiments described herein and many of the attendant advantages thereof may be readily obtained by reference to the following detailed description when considered with the accompanying drawings, wherein:

FIG. 1 is a perspective view of the syringe cartridge system in accordance with one or more illustrative embodiments of the present disclosure;

FIG. 2 is an exploded perspective view of the syringe cartridge system illustrating the plunger, the compressible cartridge and the syringe housing in accordance with one or more illustrative embodiments of the present disclosure;

FIG. 3 is a side elevation view of the syringe cartridge system in accordance with one or more illustrative embodiments of the present disclosure;

FIG. 4 is a side cross-sectional view of the syringe assembly in accordance with one or more illustrative embodiments of the present disclosure;

FIG. 5 is a perspective view of the plunger of the syringe cartridge system in accordance with one or more illustrative embodiments of the present disclosure;

FIGS. 6 and 7 are first and second side elevation views of the plunger of the syringe cartridge system in accordance with one or more illustrative embodiments of the present disclosure;

FIG. 8 is a top plan view of the plunger of the syringe cartridge system in accordance with one or more illustrative embodiments of the present disclosure;

FIGS. 9 and 10 are first and second side elevation views of the compressible cartridge of the syringe cartridge system in accordance with one or more illustrative embodiments of the present disclosure;

FIG. 11 is a top plan view of the compressible cartridge of the syringe cartridge system in accordance with one or more illustrative embodiments of the present disclosure;

FIG. 12 is a side elevation view of another illustrative compressible cartridge of the syringe cartridge system in accordance with one or more illustrative embodiments of the present disclosure;

FIG. 13 is a side elevation view of the syringe housing of the syringe cartridge system in accordance with one or more illustrative embodiments of the present disclosure;

FIG. 14 is a side cross-sectional view of the syringe housing of the syringe cartridge system in accordance with one or more illustrative embodiments of the present disclosure;

FIG. 15 is a top plan view of the syringe housing of the syringe cartridge system in accordance with one or more illustrative embodiments of the present disclosure;

FIG. 16 is an enlarged view of the area of detail identified in FIG. 14 of the syringe housing in accordance with one or more illustrative embodiments of the present disclosure;

FIGS. 17 and 18 are views illustrating insertion of the compressible cartridge into the syringe housing in accordance with an exemplative methodology of use of the syringe cartridge system in accordance with one or more illustrative embodiments of the present disclosure;

FIGS. 19 and 20 are views illustrating the plunger and the compressible cartridge disposed within the syringe housing corresponding to an unactuated condition of the plunger in accordance with one or more illustrative embodiments of the present disclosure;

FIG. 21 is an enlarged isolated view in cross-section illustrating the compressible cartridge engaged with the annular shelf of the syringe housing corresponding to the unactuated condition of the plunger in accordance with one or more illustrative embodiments of the present disclosure;

FIG. 22 is a view illustrating the plunger advanced within the syringe housing corresponding to activated condition of the plunger in accordance with one or more illustrative embodiments of the present disclosure;

FIG. 23 is an enlarged isolated view in cross-section illustrating the compressible cartridge engaged with the annular rib of the syringe housing to maintain the compressible cartridge in a compressed condition in accordance with one or more illustrative embodiments of the present disclosure;

FIGS. 24A-24C are views of alternate embodiments of anti-activating mechanism(s) preventing activation or movement of the plunger within the syringe housing of the syringe cartridge system in accordance with one or more illustrative embodiments of the present disclosure;

FIGS. 25 and 26 are views of alternate embodiments of anti-activating mechanism(s) preventing activation or movement of the plunger within the syringe housing of the syringe cartridge system in accordance with one or more illustrative embodiments of the present disclosure;

FIG. 27 is a perspective view of a medical tray apparatus in accordance with one or more illustrative embodiments of the present disclosure;

FIG. 28 is an exploded perspective view of the medical tray apparatus illustrating the base tray, the cartridges stored in the base tray, a seal for enclosing the cartridges in the base tray and a cover in accordance with one or more illustrative embodiments of the present disclosure;

FIGS. 29 and 30 are top and bottom perspective views respectively of the base tray of the medical tray apparatus in accordance with one or more illustrative embodiments of the present disclosure;

FIG. 31 is a bottom plan view of the base tray of the medical tray apparatus in accordance with one or more illustrative embodiments of the present disclosure;

FIG. 32 is a side elevation view of the base tray of the medical tray apparatus in accordance with one or more illustrative embodiments of the present disclosure;

FIG. 33 is a cross-sectional view of the base tray of the medical tray apparatus along the lines B-B of FIG. 32 in accordance with one or more illustrative embodiments of the present disclosure;

FIG. 34 is an enlarged view of the area of detail identified in FIG. 33 of the base tray of the medical tray apparatus in accordance with one or more illustrative embodiments of the present disclosure;

FIGS. 35 and 36 are top and bottom perspective views respectively of the base tray and the seal of the medical tray apparatus in accordance with one or more illustrative embodiments of the present disclosure;

FIG. 37 is an enlarged perspective view of a portion of the base tray of the medical tray apparatus in accordance with one or more illustrative embodiments of the present disclosure;

FIG. 38 is a perspective view of first and second base trays of the medical tray apparatus in stacked relation in accordance with one or more illustrative embodiments of the present disclosure;

FIG. 39 is an enlarged perspective view of a portion of the medical tray apparatus in accordance with one or more illustrative embodiments of the present disclosure;

FIG. 40 is a perspective view of first and second medical tray apparatuses in stacked relation in accordance with one or more illustrative embodiments of the present disclosure;

FIG. 41 is a perspective view of another base tray of the medical tray apparatus in accordance with one or more illustrative embodiments of the present disclosure; and

FIG. 42 is a top plan view illustrating multiple strips of multiple medical tray apparatuses to be arranged in coupling relation in accordance with one or more illustrative embodiments of the present disclosure.

### DETAILED DESCRIPTION

Embodiments described herein provide systems and methods for pre-filled medical delivery assemblies that provide advantages and benefits as described herein. For example, the syringe cartridge assembly of some embodiments may include a Blow-Fill-Seal (BFS) vial or bottle module couplable to a syringe housing. In some embodiments, such a syringe cartridge assembly may be selectively activated by application of a longitudinal force to one of the delivery member/needle or syringe housing, causing the delivery member/needle to axially advance and pierce a fluid reservoir of the BFS vial module. Utilization of such systems that employ BFS vials may be advantageous and may address various shortcomings of previous systems.

BFS vials may, for example, offer a less expensive alternative to typical vials or devices created via other manufacturing techniques. In some embodiments, BFS vials (e.g., due to the nature of the BFS manufacturing process) may not require separate sterilization (e.g., an may accordingly be compatible with a wider array of fluids), may provide enhanced production rates of sterile/aseptic units per hour, and/or may be provided to an end-user for significantly lower per dose/unit costs. In some embodiments, BFS vials reduce the need to meet precise manufacturing tolerances and may be readily adaptable to form certain mating features such as standardized threads.

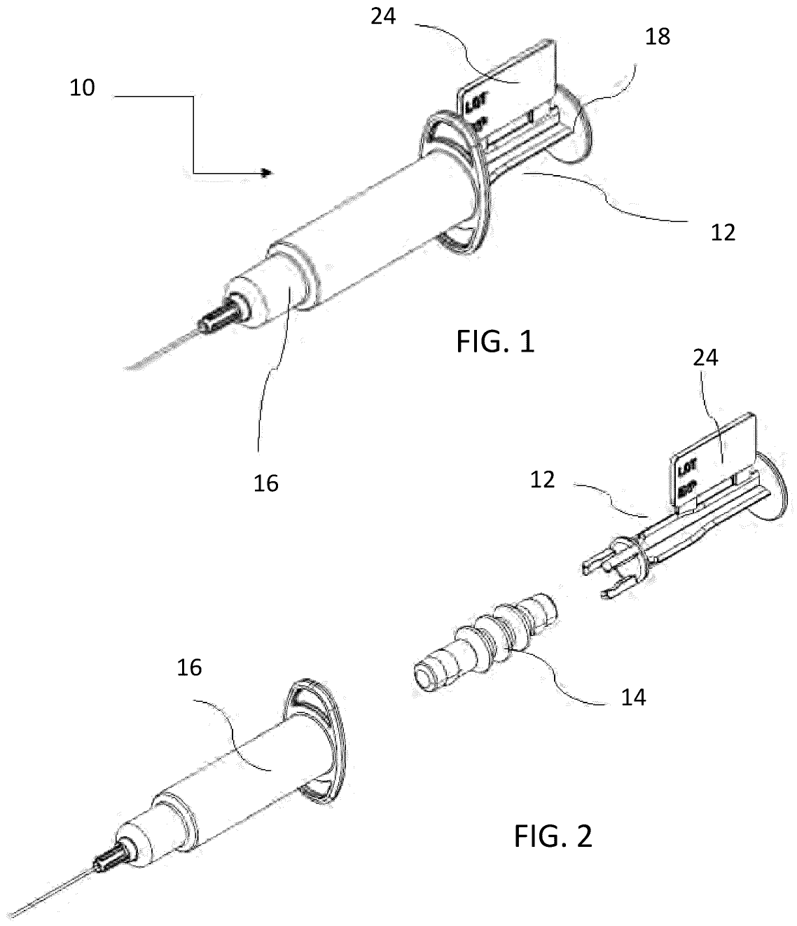

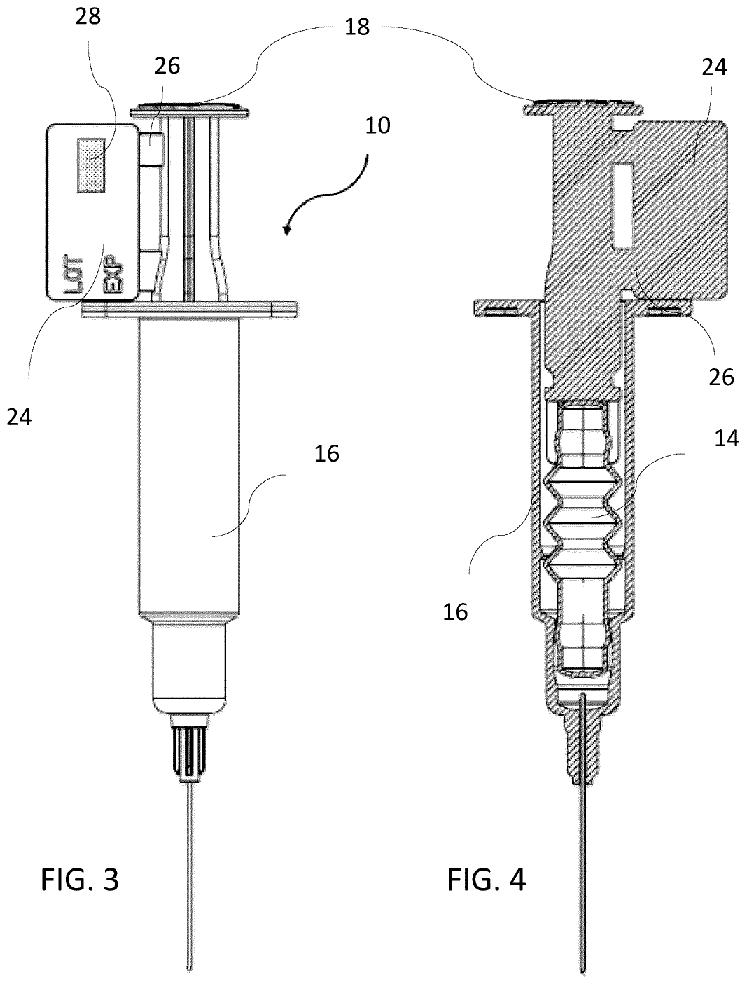

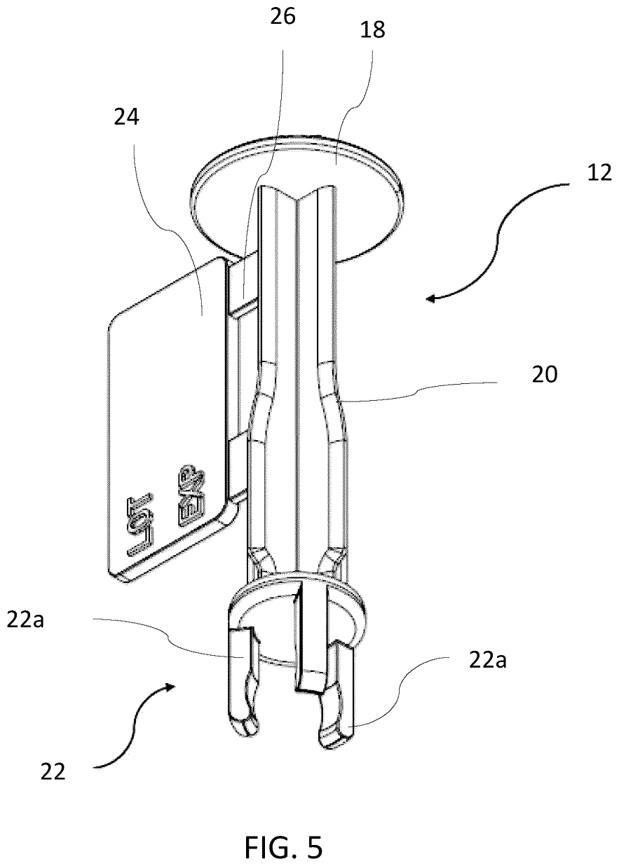

Referring now to FIGS. 1-4, there is illustrated one exemplative embodiment of the syringe cartridge assembly according to the present disclosure. The syringe cartridge assembly 10 generally includes three components, from proximal to distal, a plunger 12, a vial or compressible cartridge 14 and a syringe housing 16 which receives the compressible cartridge 14 and the plunger 12. As depicted in FIGS. 3-5, in conjunction with FIGS. 1 and 2, the plunger 12 includes a disc-shaped handle 18 (although any geometric shape or configuration for such handle may be used), an elongate plunger element 20 and a grasper segment or component 22 at the end of the plunger element 20. The plunger 12 may further include a flag segment 24 depending outwardly from the plunger element 20. The plunger 12 may be fabricated from a suitable polymeric material and formed via known methodologies including injection molding techniques or the like. The grasper component 22 is optional and may be in the shape of a claw or the like. The grasper component 22 may include one or more grasping legs 22a, for example, three legs 22a, radially spaced about an axis of the plunger 12. The grasping legs 22a may have contoured interior surfaces which assist in engaging the compressible cartridge 14 to lift the compressible cartridge 14 from (for example, from a placard or container holding multiple compressible cartridges 14) for placement within the syringe housing 16. The flag segment 24 functions as an anti-activating mechanism preventing activation or movement of the plunger 12 within the syringe housing 16 until it is detached, folded or otherwise removed or repositioned, for example, along score or break segments 26 connecting the flag segment 24 to the plunger element 20. More specifically, the flag segment 24 prevents the compressible cartridge 14 from being pushed down into the syringe housing 16 and pierced by the needle of the housing 16 until it is removed or displaced. (i.e., the flag segment 24, in its first or “pre-activation” position, prevents an injection of the fluid agent from being initiated; it is only when the flag segment 24 is removed or placed into a second “activation” position (repositioned, etc.) that the injection of the fluid agent may be initiated.

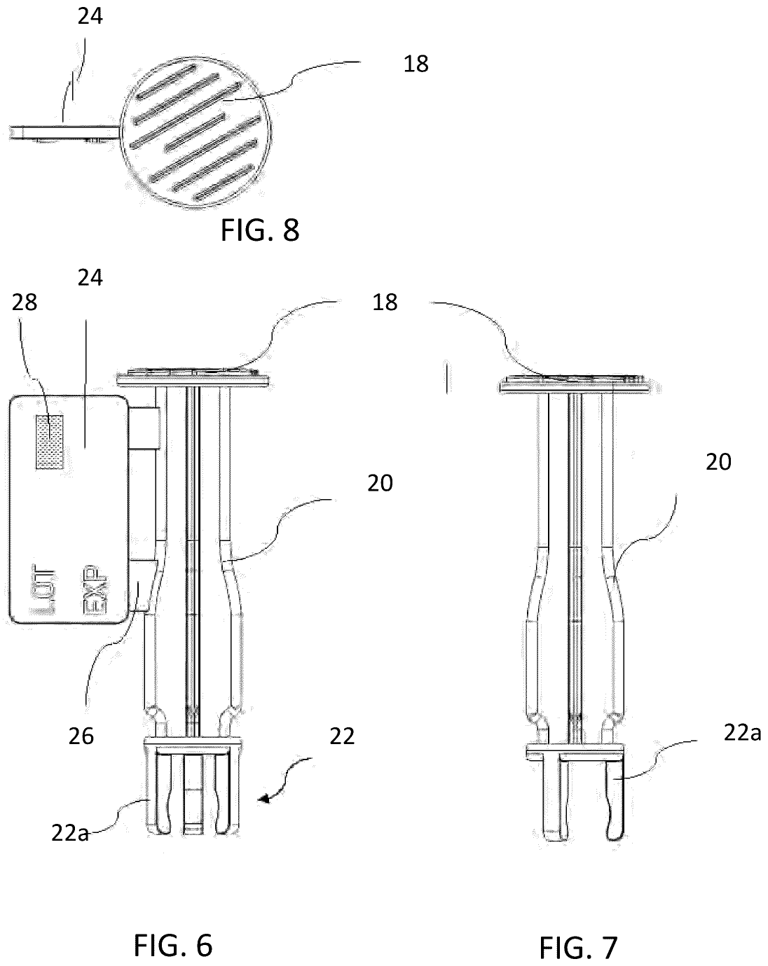

In accordance with some embodiments, the plunger 12 may further include information and/or data storage mechanisms that uniquely identify each plunger or syringe cartridge assembly 10. Such information and/or data storage mechanisms may include human-readable and/or machine-readable information (e.g., in the form of one or more codes). For example, such data storage mechanisms may comprise, a Near Field Communication (NFC) chip, a Radio Frequency Identification (RFID) chip, a QR code, a bar code or any other machine-readable identifier or indicia, identified as reference numeral 28 (FIG. 6), that allows for a unique identifier of an syringe assembly and/or component thereof (e.g., the plunger assembly, as illustrated in FIG. 6) to be stored in association therewith (e.g., attached to, engraved on, coupled to or embedded in, the syringe assembly or a portion thereof, such as on or underneath a label on a label-portion thereof). One exemplary data storage system utilizing identifiers or indicia for BFS fluid agent delivery devices is disclosed in commonly assigned PCT Application No. PCT/US21/30530, filed May 3, 2021 and titled MEDICAL INJECTORS AND SYSTEMS AND METHODS FOR AN INJECTION MANAGEMENT PLATFORM, the entire contents of which is incorporated by reference herein.

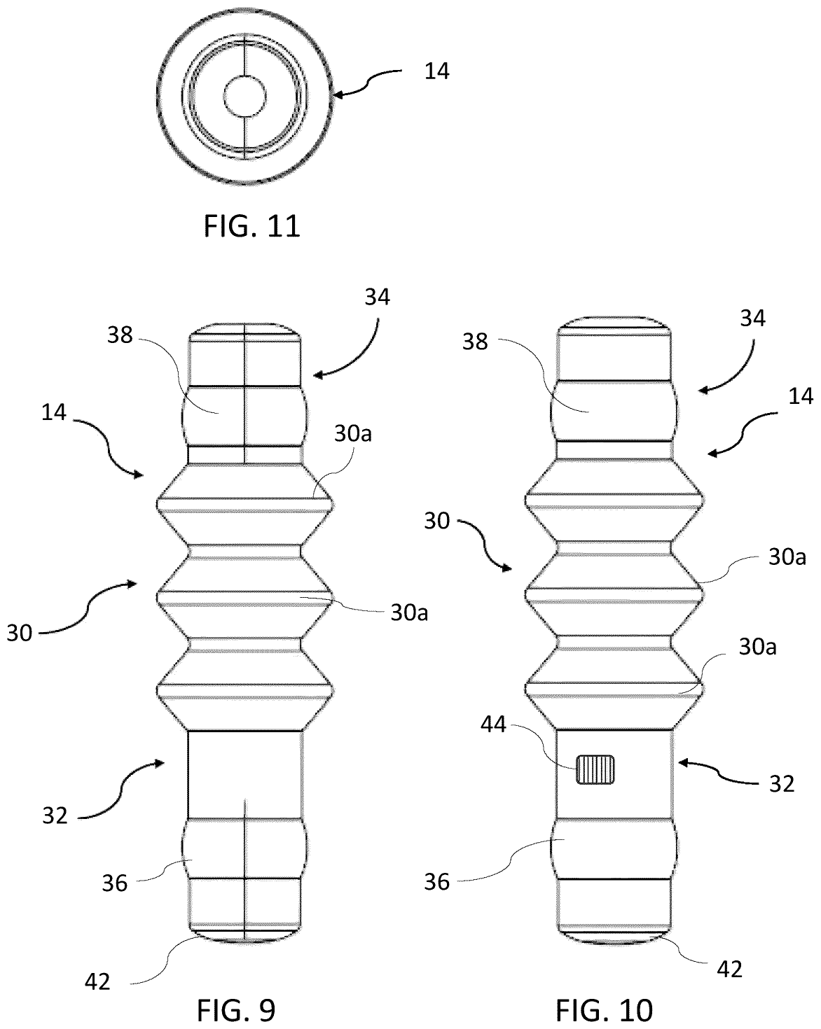

Referring now to FIGS. 9-11, in view of FIGS. 1-4, the compressible cartridge 14 will be discussed. The compressible cartridge 14 is the vial or vial source that holds the fluid agent to be delivered to a patient. The compressible cartridge 14 may be fabricated using Blow-Fill-Seal (BFS) technology. BFS technology is a manufacturing technique used to produce small, (0.1 mL) and large volume, (500 mL +) liquid-filled containers. During the BFS manufacturing process the compressible cartridge 14 is formed, filled, and sealed in a continuous process without human intervention, in a sterile enclosed area inside a machine. In particular, the BFS manufacturing process is multi-step one in which a pharmaceutical-grade plastic resin is vertically heat extruded through a circular opening to form a hanging tube called the parison. This extruded tube is then enclosed within a two-part mold, and the tube is cut above the mold. The mold is transferred to the filling zone, or sterile filling space, where filling needles (mandrels) are lowered and used to inflate the plastic to form the container within the mold. Following the formation of the compressible cartridge 14, the mandrel is used to fill the compressible cartridge 14 with liquid. Following filling, the mandrels are retracted and a secondary top mold seals the container. All actions take place inside a sterile shrouded chamber inside the machine. The product is then discharged to a non-sterile area for labeling, packaging and distribution.

In illustrative embodiments, the compressible cartridge 14 includes a single dose of the fluid agent or, optionally, may include multiple doses. In accordance with some embodiments, the compressible cartridge 14 includes a central bellows or accordion structure 30 including multiple bellows or folds 30a (three are shown, but the embodiments described herein may be adapted to other numbers of bellows) configured to contract and expand or widen within the syringe housing 16 upon compression of the compressible cartridge 14 along its axis. The contraction of the bellows structure 30 provides sufficient compressive forces to maximize expulsion of the fluid agent from the compressible cartridge 14. In one illustrative embodiment, compression of the multiple bellows 30a corresponds to dispensing of a single dose of a fluid agent, and exhaustion of the fluid agent. In another illustrative embodiment, compression of one or more individual bellows 30a corresponds to delivery of one dose of the fluid agent while additional fluid agent remains in the compressible cartridge 14 for one or more additional doses, i.e., multiple doses. Continued compression of the multiple individual bellows 30a will release the additional doses in sequence. It should be noted that other configurations of BFS vials that are compressible may be used, and the embodiments described herein are not dependent on a bellows structure of a cartridge.

On opposed sides of the bellows structure 30 is a proximal neck segment 32 and a distal neck segment 34, which are narrower or define a smaller cross-sectional dimension than the bellows structure 30. In the illustrative embodiment depicted in FIGS. 9-11, the distal neck segment 34 defines a greater length than the length of the proximal neck segment 32 along the axis of the compressible cartridge 14. The asymmetric arrangement of the proximal and distal neck segments 32, 34 of the compressible cartridge 14 illustrated in FIGS. 9-11 assists the user in correctly positioning the compressible cartridge 14 in the proper direction within the syringe housing 16, i.e., with the distal neck segment 34 being first introduced and advanced within the syringe housing 16.

The distal neck segment 34 of the compressible cartridge 14 defines a distal outward annular donut or collar 36 spaced from the extreme remote end of the compressible cartridge 14. The distal collar 36 cooperates with corresponding geometry within the syringe housing 16 to provide tactile feedback (e.g., a snap-in feel) that the compressible cartridge 14 has been advanced and is properly positioned within the syringe housing 16 for activation. In illustrative embodiments, the distal collar 36 cooperates with the corresponding structure within the syringe housing 16 to prevent removal of the compressible cartridge 14 once properly positioned (in an initial position or condition prior to activation) within the syringe housing 16. The proximal neck segment 32 also may include a proximal donut or collar 38 for coupling with the grasper component 22 of the plunger 12. More specifically, the grasping legs 22a are arranged to receive the proximal collar 38 in releasable secured relation therewith. The remote end 42 of the compressible cartridge 14 defines an arcuate or convex shape which facilitates wiping and/or sterilization prior to use. The remote end 42 is pierced by the needle of the syringe housing 16. In addition, the compressible cartridge 14 also may include visual indicia, identified as reference numeral 44, such as an RFID tag or bar code, to facilitate identification of the treatment agent or compressible cartridge type or size as described hereinabove.

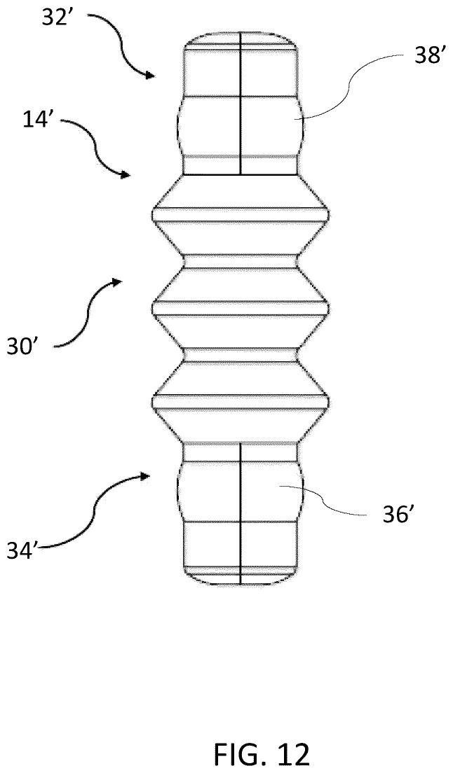

FIG. 12 illustrates a compressible cartridge 14′where the proximal and distal neck segments 32′, 34′define substantially the same length. In other respects the compressible cartridges 14, 14′may be substantially similar.

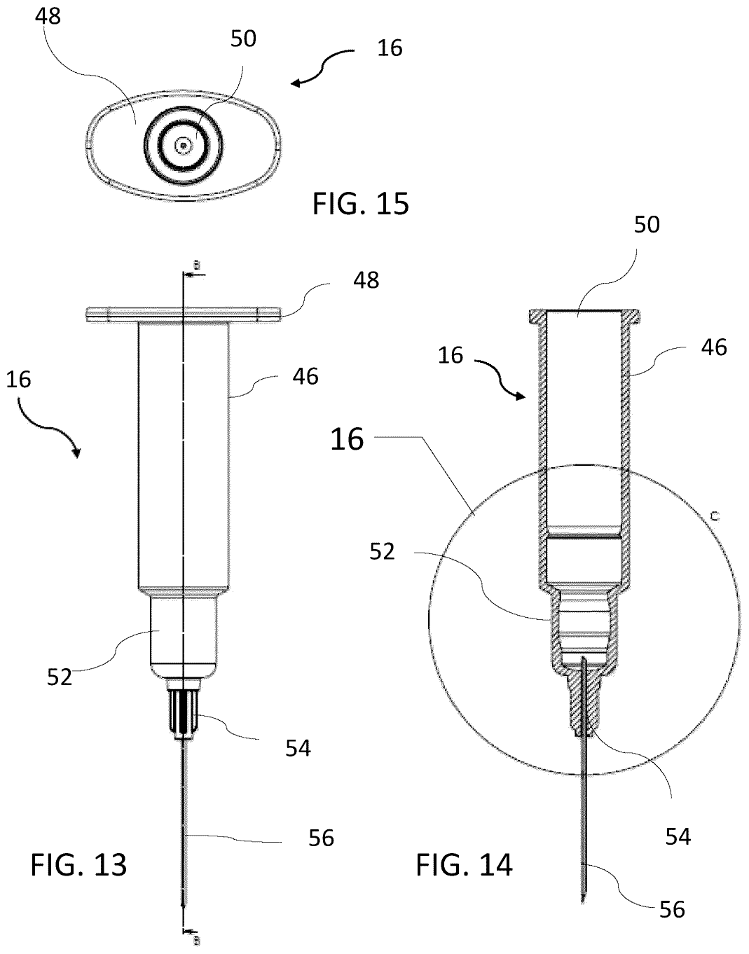

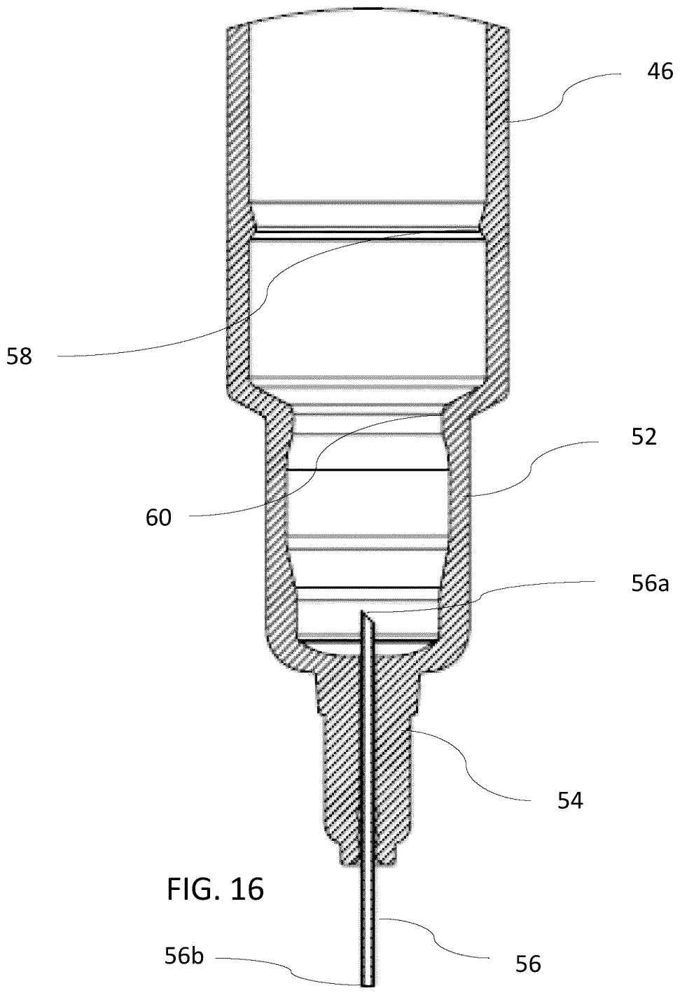

Referring now to FIGS. 13-16, in conjunction with the view of FIGS. 1-4, the syringe housing 16 will be discussed. The syringe housing 16 includes an open main syringe body 46 having flange 48 defining an opening 50 extending into the internal chamber of the syringe body 46. The main syringe body 46 leads to a narrow syringe neck 52 and to a needle hub 54. The needle hub 54 houses a cannulated needle 56 (e.g., a delivery member) which is secured within the needle hub 54 via adhesives or the like. As best depicted in the enlarged view of FIG. 16, in the interior of the main syringe body 46 is an at least partial annular rib 58. In illustrative embodiments, the annular rib 58 prevents removal and/or reuse of the compressible cartridge 14 through engagement with one or more of the individual compressed bellows or folds 30a of the bellow structure 30 in accordance with one illustrative embodiment of an auto-disabling feature as will be described hereinbelow, e.g., when the compressible cartridge 14 is in, or transitions to, a compressed condition. In embodiments, the annular rib 58 may be a partial rib or rib segment extending for a portion of the internal dimension of the main syringe body 46. In some embodiments, the annular rib 58 is a full rib extending along a major portion of, or the entire, internal or inner dimension of the main syringe body 46. Multiple partial ribs or full ribs are also contemplated.

The syringe neck 52 includes an at least partial annular shelf 60 adjacent the juncture of the main syringe body 46 and the syringe neck 52. In illustrative embodiments, the annular shelf 60 cooperates with the distal collar 36 of the distal neck segment 34 of the compressible cartridge 14 to secure the compressible cartridge 14 within the syringe housing 16. In other illustrative embodiments, the annular shelf 60 cooperates with the distal collar 36 to provide a tactile indicator to the user that the compressible cartridge 14 is appropriately positioned within the syringe housing 16 and ready to be activated. In certain embodiments, the annular shelf 60 prevents removal and/or reuse of the compressible cartridge 14 in accordance with an alternative disabling feature of the syringe cartridge assembly 10. In embodiments, the annular shelf 60 may be a partial shelf segment extending for a portion of the internal dimension of the main syringe body 46 or may be a full shelf extending along the entire internal dimension of the main syringe body 46. Multiple partial shelves are also contemplated.

The cannulated needle 56 is a double ended needle and extends within the interior of the syringe neck 52 at one end and beyond the needle hub 54 at the second end. The needle end 56a within the syringe neck 52 is dimensioned to pierce the remote end 42 of the compressible cartridge 14 and the needle end 56b is dimensioned to pierce the subject or patient. The needle ends 56a, 56b may be straight, beveled, multi-beveled, etc. One suitable needle arrangement for use with the syringe cartridge assembly 10 is described in commonly assigned PCT Application Serial No. PCT/US21/61991, filed Dec. 6, 2021 and titled SYSTEMS AND METHODS FOR ROTATIONAL PIERCING OF PRE-FILLED MEDICAL DELIVERY ASSEMBLIES, the entire contents of which is incorporated by reference herein.

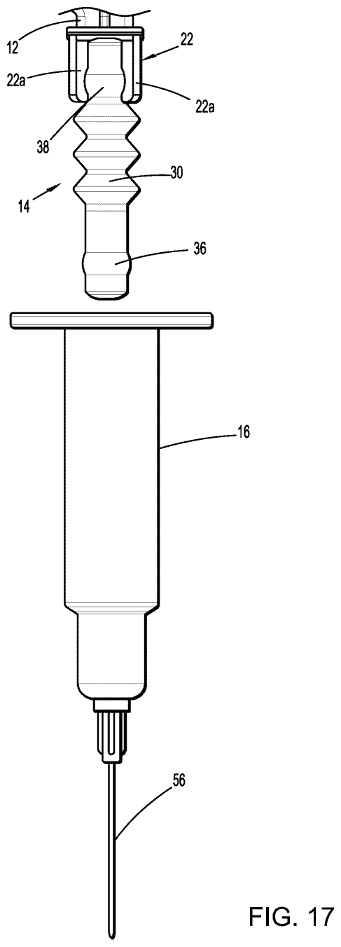

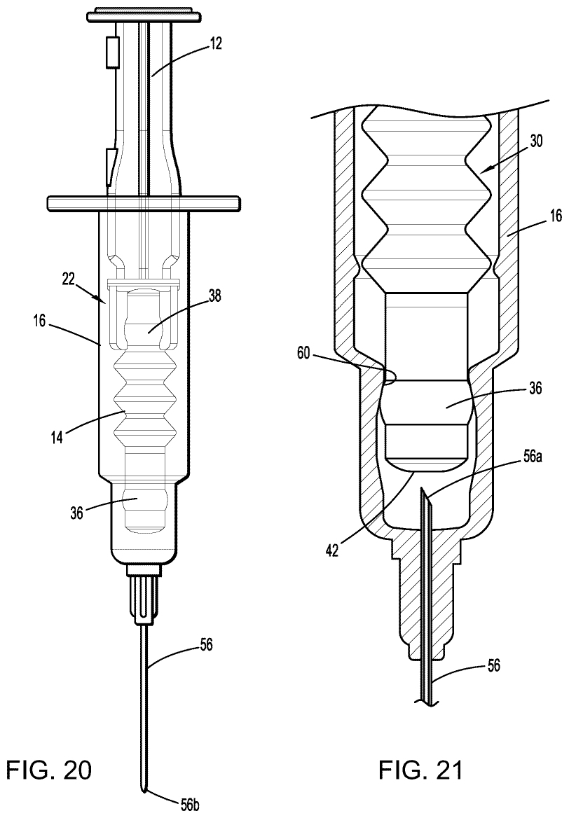

FIGS. 17-23C illustrate an exemplative methodology of use of the syringe cartridge assembly 10 in accordance with one or more embodiments of the present disclosure. In FIG. 17, the plunger 12, independent of the other components of the syringe cartridge assembly 10, is depicted engaged with an individual compressible cartridge 14. In illustrative embodiments, the plunger 12 may be advanced, either manually or through automated mechanism(s), toward an aligned compressible cartridge 14 whereby the grasper component 22 engages the compressible cartridge 14. In illustrative embodiments, the grasping legs 22a of the grasper component 22 engage the proximal collar 38 of the proximal neck segment 32 of the compressible cartridge 14 (FIGS. 9 and 10) in secured relation therewith as facilitated via the contoured surfaces of the grasping legs 22a. In certain embodiments, the proximal collar 38 compresses to enable positioning of the grasping legs 22a about the proximal collar 38. In other embodiments, the grasping legs 22a may deflect outwardly to accommodate the proximal collar 38. A combination of compression of the proximal collar 38 and outward movement of the grasping legs 22a is also envisioned. In other embodiments, the grasper component 22 may not engage the proximal collar 38 but rather directly engage another segment of the proximal neck segment 32.

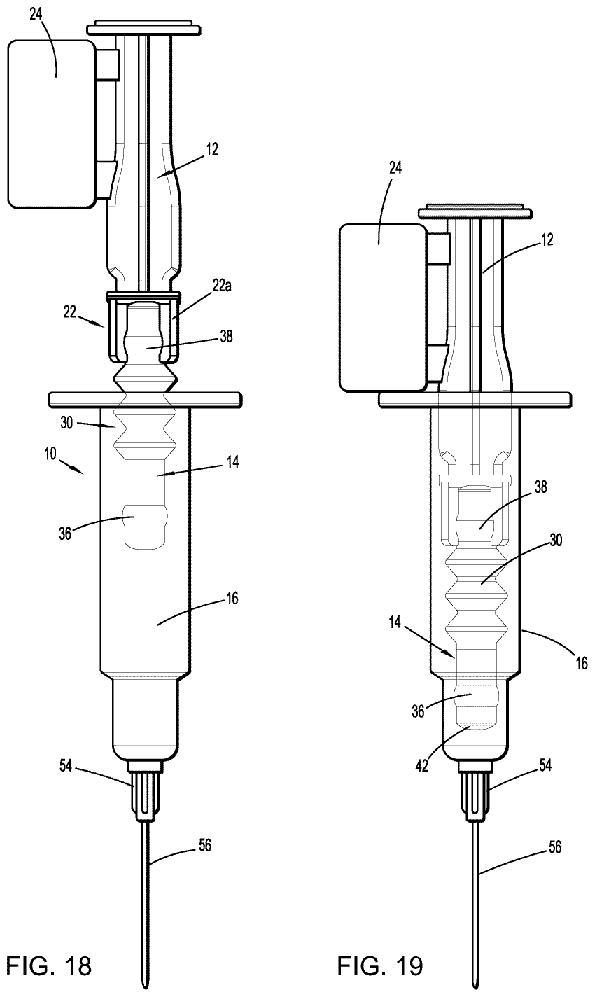

Referring now to FIG. 18, the plunger 12 is maneuvered to introduce the compressible cartridge 14 within the syringe housing 16. The plunger 12 is continually advanced to the position depicted in FIGS. 19-21 in which the distal collar 36 of the distal neck segment 34 of the compressible cartridge 14 (FIGS. 9 and 10) engages and passes the annular shelf 60 within the syringe neck 52 of the syringe housing 16 (FIG. 16). Upon clearance of the distal collar 36, the user is provided with a tactile indicator that the compressible cartridge 14 is mounted within the syringe housing 16 in position to be activated. This corresponds to the first, retracted or unactuated condition of the plunger 12 (i.e., in an initial or inactivated condition but ready for use). In addition, upon clearance of the distal collar 36 of the compressible cartridge 14 relative to the annular shelf 60, the compressible cartridge 14 may be secured within the syringe housing 16 in a manner preventing removal via, for example, retraction of the plunger 12. In particular, in illustrative embodiments, the force(s) required for the distal collar 36 to clear the annular shelf 60 within the syringe housing 16 during a retraction of the plunger 12 may be greater than the force(s) required to release the grasper component 22 of the plunger 12 from the proximal collar 38 of the compressible cartridge 14, thus causing the grasper component 22 to release the compressible cartridge 14 during this, or responsive to, retracting movement of the plunger 12. Thus, in illustrative embodiments, the disabling feature will prevent removal or backing out of the compressible cartridge 14 once in the initial condition of the compressible cartridge 14.

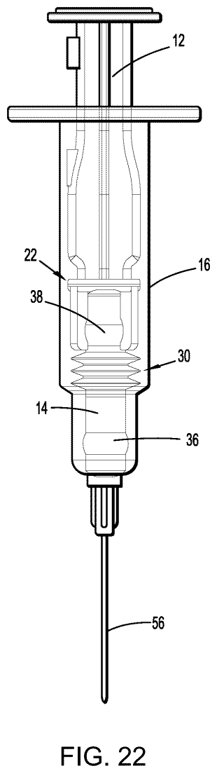

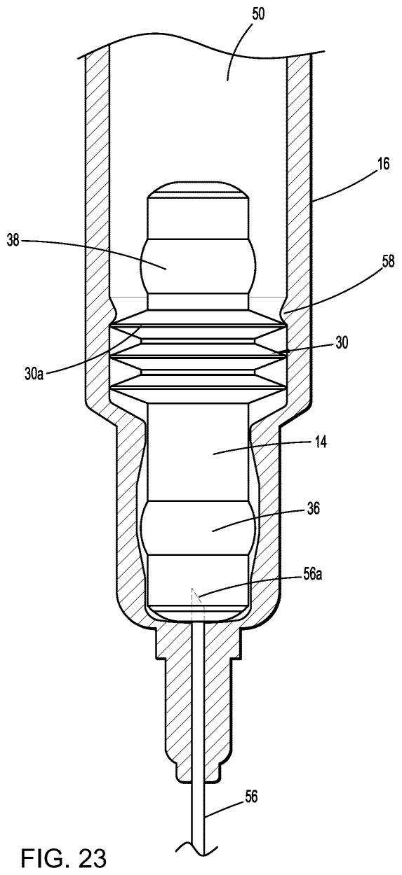

The process is continued by removing or repositioning the flag segment 24 relative to the plunger 12. With the flag segment 24 removed or repositioned, the plunger 12 may be advanced as depicted in FIG. 22 to a second, activated or advanced position to compress the bellow structure 30 whereby the needle end 56a of the cannulated needle 56 penetrates the penetrable remote end 42 of the compressible cartridge 14 to enable the fluid agent to be dispensed through the cannulated needle 56 and into the subject or patient. As noted above, in illustrative embodiments, once the compressed bellow structure 30 clears the annular rib 58 of the syringe housing 16 as shown in FIG. 23, the user may be provided with a tactile indicator that the treatment fluid has been delivered to the patient. In addition, the cooperative engagement of the annular rib 58 of the syringe housing 16 with the bellow structure 30, for example, the trailing or proximal individual bellow 30 of the bellow structure 30 may serve to prevent removal of the compressible cartridge 14 from the syringe housing 16 subsequent to activation. In particular, in illustrative embodiments, the force(s) required for the bellow structure 30 to clear the annular rib 58 of the syringe housing 16 may be greater than the force(s) required to release the grasper component 22 of the plunger 12 from the proximal collar 38 of the compressible cartridge 14, thus causing the grasper component 22 to release the compressible cartridge 14 during a retracting movement of the plunger 12. Thus, the compressible cartridge 14 may not be reused. In addition, the compressible cartridge 14 may not reopen which would potentially undesirably draw (under vacuum) blood or other fluids back into the compressible cartridge 14. FIG. 23 illustrates the plunger 22 removed from the compressible cartridge 14 subsequent to release of the grasper component 22 from the proximal collar 38 of the compressible cartridge 14.

In certain embodiments, the compressible cartridge 14 may accommodate multiple doses. For example, and without limitation, one dose may correspond to a first individual bellow of the bellows structure 30 clearing the annular rib 58 and subsequent doses may correspond to the additional individual bellows clearing the annular rib 58 in sequence.

In other embodiments, the plunger 22 is devoid of the grasper component 22. For example, in some embodiments a user may use other means of placing a compressible cartridge 14 into the syringe housing 16 (or may come pre-assembled within the syringe housing 16) and the plunger 22 may be utilized to compress the cartridge 14 but not to grasp it.

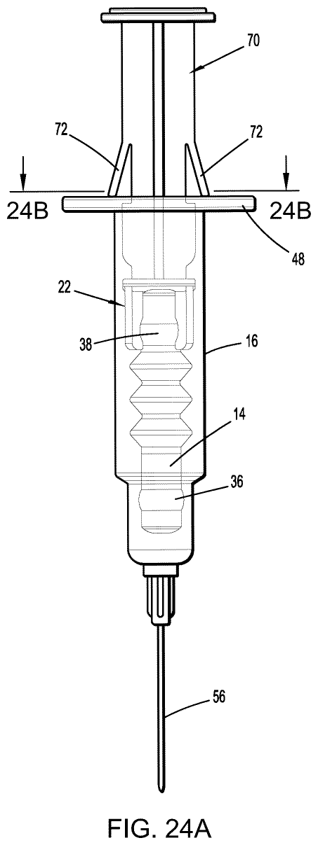

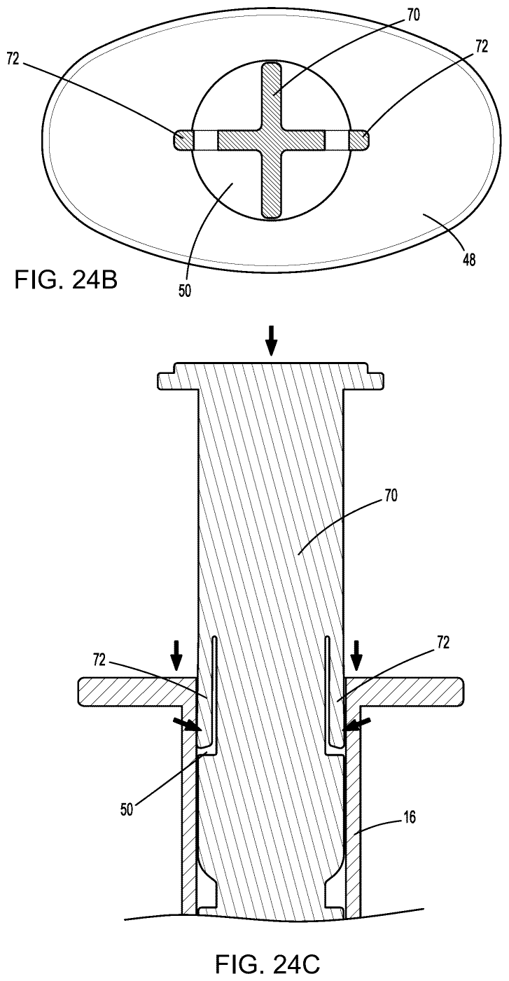

FIGS. 24A-24C illustrate another embodiment of a plunger 70 for use with the syringe cartridge assembly 10. In lieu of the flag segment 24, the plunger 70 includes opposed deflectable arms 72 as the anti-activating mechanism. In embodiments, the deflectable arms 72 may be monolithically formed with the plunger 70 and disposed in cantilevered relation with the main body of the plunger 70. The deflectable arms 72 are normally arranged in oblique relation to the axis of the plunger 70 in position to engage the flange 48 of the syringe housing 16 to maintain the plunger 70 in an initial unactuated position. When the plunger 70 is ready to be activated, the deflectable legs 72 may be depressed radially inwardly to be aligned with the opening 50 of the syringe housing 16. In this position of the deflectable legs 72, the plunger 70 may be advanced within the syringe housing 16 to compress the compressible bladder 14.

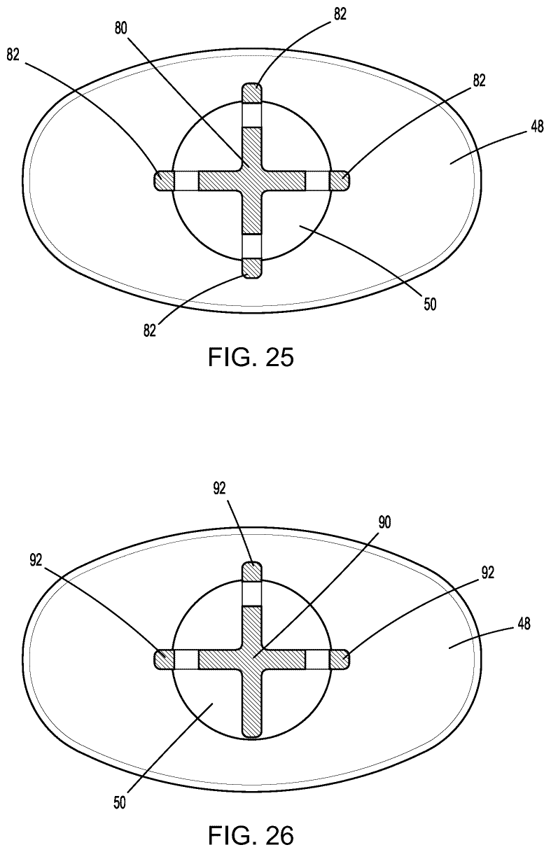

FIG. 25 illustrates, in a schematic top plan view, another embodiment of anti-activation mechanism for a plunger for use with the syringe cartridge assembly 10. The anti-activation mechanism replaces the flag segment 24 and includes one or more deflectable arms 82 coupled to the plunger element 80. The deflectable arms 82 are arranged in a “t” shape or a cross shape in a normal condition of the deflectable arms 82, and may be depressed radially inwardly, individually or collectively, to define a dimension less than the dimension of the opening 50 of the syringe housing 16. In embodiments, the one or more deflectable arms 82 are disposed in cantilever relation with the main body of the plunger 80, and monolithically formed therewith. In the displaced condition, the deflectable arms 82 will clear the opening 50 when depressed inwardly to permit advancement of the plunger 80 through the syringe housing 16. FIG. 26 illustrates an anti-activation mechanism where the plunger element 90 includes three deflectable legs 92 which may be depressed radially inwardly individually or collectively relative to the plunger element 90.

The syringe cartridge assembly embodiments described herein offer various advantages resulting from the design. For example, the system is designed to be used with various types of needles or injections (e.g., intradermal, subcutaneous or intramuscular). As another example, the system can readily be sized for a wide variety of volumes of fluid agents (e.g., small doses such as 0.5 mL or large doses such as 5 mL or 10 mL that may be used for larger animals or to administer infusions by syringe).

Other embodiments may also include additional advantages to enhance the syringe cartridge assembly. For example, one or more “pairing safeguards” may be incorporated into the system design to assist a user in matching an appropriate plunger 12 to a compressible cartridge 14 holding an appropriate fluid agent and/or dose (e.g., the grasper structure on a plunger 12 that is intended for use only with certain vaccines may be color coded and/or shaped to key into or fit only with a matching color and/or shape of a compressible cartridge 14 that has a matching shape and/or color on the neck portion that the grasper structure mates to; a similar shape-key safeguard mechanism may be utilized to assist a user in picking up a children's dosage cartridge only with a plunger that includes a grasper structure having a matching corresponding shape and/or color.

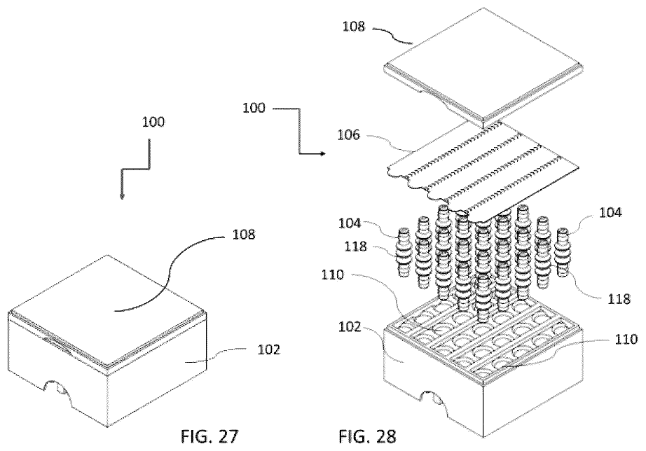

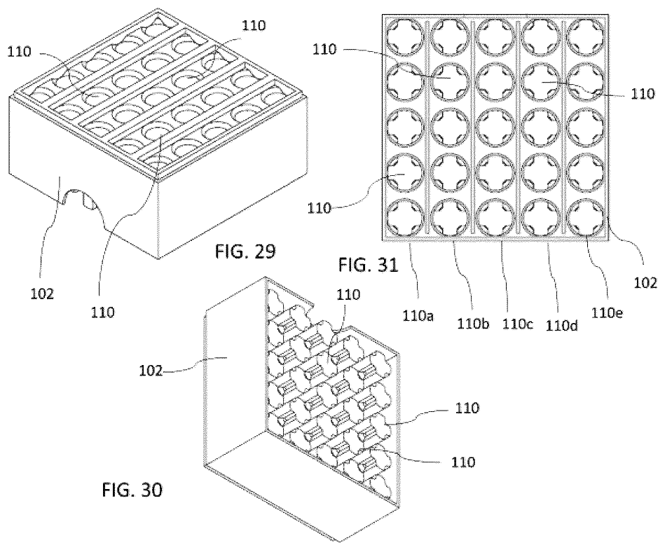

Referring now to FIGS. 27 and 28, there is illustrated an exemplative embodiment of a medical tray apparatus according to the present disclosure. The medical tray apparatus 100 generally includes at least some of the four following components: a base tray 102, a plurality of individual cartridges 104 at least partially accommodated by the base tray 102, a seal 106 positionable over the individual cartridges 104 and a lid or cover 108 mountable to the base tray 102. As depicted in FIGS. 29-33, in conjunction with FIGS. 27 and 28, the base tray 102 includes a plurality of individual cartridge receptacles 110 arranged, in illustrative embodiments, in rows 110a-110e with each row 110a-110e having a defined number of individual cartridge receptacles 110. Although five rows 110a-110e are illustrated with each row 110a-110e containing five individual cartridge receptacles 110, the base tray 102 may have more or less than five rows and a different number of individual cartridge receptacles 110, including, for example, and without limitation, three rows containing four individual cartridge receptacles 110, etc. Other variations are also contemplated. The individual cartridge receptacles 110 each have an internal wall defining a central opening for reception of an individual cartridge 104.

The base tray 102 may be fabricated from a suitable polymeric material manufactured via known molding techniques to contain the multiple individual cartridge receptacles 110. In some embodiments, the base tray 102 incorporates or comprises an insulative material such as a polystyrene, a polyurethane foam or the like. In other embodiments, the base tray 102 may be made from a metal material and/or combinations of a polymeric material and a metal material. The insulative material may be integrally formed with the base tray 102, for example, incorporated within the walls of the base tray 102, disposed in the underside of the base tray 102 and/or surround the multiple individual cartridge receptacles 110 of the base tray 102.

In other embodiments, the base tray 102 includes a cooling medium. The cooling medium may be integrally formed with the base tray 102, disposed between multiple layers forming the base tray 102, disposed in a reservoir surrounding the base tray 102 and/or surrounding the cartridge receptacles 110. The cooling medium may include water (in, e.g., in the form of cooled water or ice), a refrigerant liquid or gel, ammonia or any other type of cooling liquid, and may include materials which may undergo an endothermic change when mixed such as ammonia nitrate and water. Other locations for the cooling medium within the medical tray apparatus 100 are also envisioned.

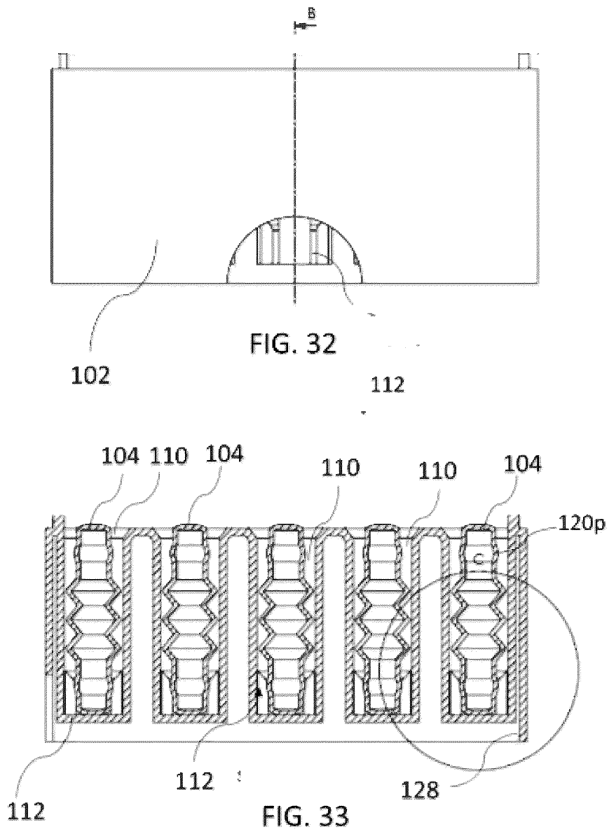

As best depicted in FIGS. 32-34, the lower end of each individual cartridge receptacle 110 includes a cartridge mount 112. The cartridge mount 112 may be integrally or monolithically formed with its respective individual cartridge receptacle 110 or be a separate component and secured within the cartridge receptable 110 via known techniques. Each cartridge mount 112 defines a tapered lead-in segment 114 terminating in lower retention segment 116. The lead-in segment 114 facilitates insertion of the individual cartridge 104 within the respective cartridge mount 112 while the retention segment 116 engages the individual cartridge 104 in secured relation therewith.

Referring again to FIGS. 27 and 28, in illustrative embodiments, the individual cartridges 104 contain a fluid agent which is to be administered to the user. The fluid agent may be a vaccine, antibiotic, therapeutic agent, vitamin(s) etc. In some embodiments, the individual cartridges 104 are compressible similar to the compressible cartridges 14 of the embodiment of FIGS. 1-26, and are fabricated using Blow-Fill-Seal (BFS) technology.

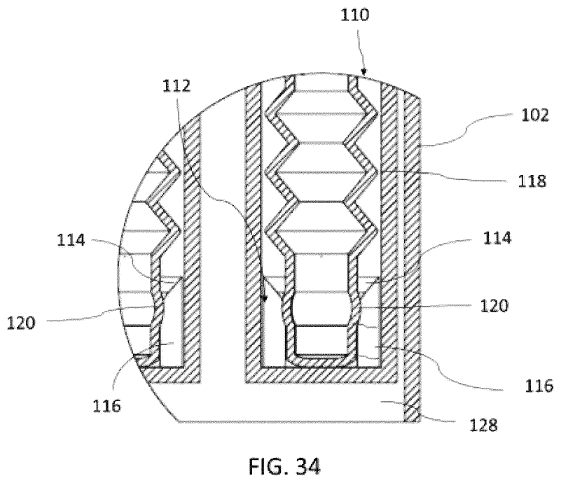

In some embodiments, the individual cartridge 104 may include a single dose of the fluid agent or, optionally, may include multiple doses. The individual cartridges 104 may include a central bellows or accordion structure 118 including multiple bellows (FIG. 34) configured to contract and expand upon compression of the individual cartridge 104 along its axis. The contraction of the bellows structure 118 provides sufficient compressive forces to maximize expulsion of the fluid agent from the compressible individual cartridge 104. In one illustrative embodiment, compression of the multiple bellows structure 118 corresponds to dispensing of a single dose of a fluid agent, and exhaustion of the fluid agent. In another illustrative embodiment, compression of one or more individual bellows of the bellow structure 118 corresponds to delivery of one dose of the fluid agent while additional fluid agent remains in the compressible individual cartridge 104 for one or more additional doses, i.e., multiple doses. Continued compression of the multiple individual bellows will release the additional doses in sequence.

As best depicted in FIGS. 33-34, the distal end of the individual cartridge 104 may define a distal outward annular donut or collar 120. The collar 120 is engaged by the retention segment 116 of the cartridge mount 112 to secure the individual cartridge 104 within the individual cartridge receptacle 110. In some embodiments, the collar 120 compresses while traversing the tapered lead-in segment 114 of the cartridge mount 112 to be secured within the retention segment 116. In some illustrative embodiments, the retention segment 116 of the cartridge mount 112 defines an opening having an internal dimension or diameter less than the outer diameter or dimension of the collar 120 of the individual cartridge 104. An individual cartridge 104 may be removed from a cartridge mounts 112 via a pulling or withdrawal motion imparted to the individual cartridge 104 with a syringe or grasping instrument away from the base tray 102. For example, in illustrative embodiments, each individual cartridge 104 may be removed via the grasper component 22 of the plunger 12 of the syringe cartridge assembly of FIGS. 1-26. The grasper component 22 may be positioned over a proximal donut or collar 120p. (FIG. 33). In embodiments, the grasping legs 22a of the grasper component may displace outwardly and/or the proximal collar 120p may compress to enable positioned of the grasper component 22 over the proximal donut to establish a secured relation with the cartridge 104 in the manner described hereinabove. In embodiments, upon removal from the cartridge mount 112, the individual cartridge 104 may provide a popping sound and/or a tactile indicator to indicate release from the cartridge mount 112.

Further details of additional compressible cartridges suitable for use with the medical tray apparatus 100 are illustrated in U.S. provisional patent Application Ser. No. 63/461,214, filed Apr. 21, 2023 and entitled “Syringe Cartridge System” and in international patent application publication number WO2017/187262 A1, published on 2 Nov. 2017 and entitled “Medical Delivery System,” the entire contents of each disclosure being incorporated by reference herein.

In some embodiments, other cartridge types having different dimensions or structure may be utilized with the medical tray apparatus 100. For example, the cartridge may be a standard vial which is compressible or non-compressible. The vial may be generally of constant dimension along its length. In some embodiments, the cartridge mount 112 may be configured and/or modified to secure multiple-type cartridges. Thus, the present disclosure is not to be interpreted as limited to the individual cartridge 104 and/or cartridge mount 112 described herein.

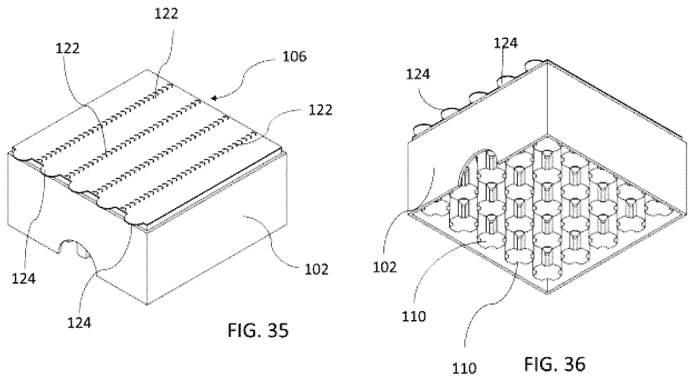

FIGS. 35 and 36 illustrate the seal 106 mounted to the base tray 102 to enclose the individual cartridges 104 within the cartridge receptacles 110. The seal 106 may be fabricated from any material suitable to maintain sterility of the contents of the base tray 102. One suitable seal 106 may be fabricated from Tyvek@ which provides microbial barrier protection for the sterilized contents of the fluid agent contained within the individual cartridges 14. Other peelable heat sealing films are also envisioned. In illustrative embodiments, the seal 106 is perforated enabling removal of portions of the seal 106 while other portions of the seal 106 remain bonded to the base tray 102. For example, the seal 106 may include one or more perforations or score lines 122 enabling access to individual rows 110a-110e (FIGS. 29-31) of the plurality of individual cartridge receptacles 110. When the individual cartridges 104 of a specific row 110a-110e are needed, a section of the seal 106 corresponding to that row may be released along the appropriate perforations or score lines 122. The seal 106 may include other arrangements of perforations enabling access to, for example, cartridge receptacles 110 on an individual basis. Other arrangements are also envisioned. In illustrative embodiments, the seal 106 includes multiple tabs 124 which extend beyond the perimeter of the base tray 102. Each tab 124 may correspond to a specific section of the seal 106 and row 110a-110e of the cartridge receptacles 110. The tabs 124 facilitate engagement by the clinician of the selected section of the seal 106 to expose the selected row 110a-110e.

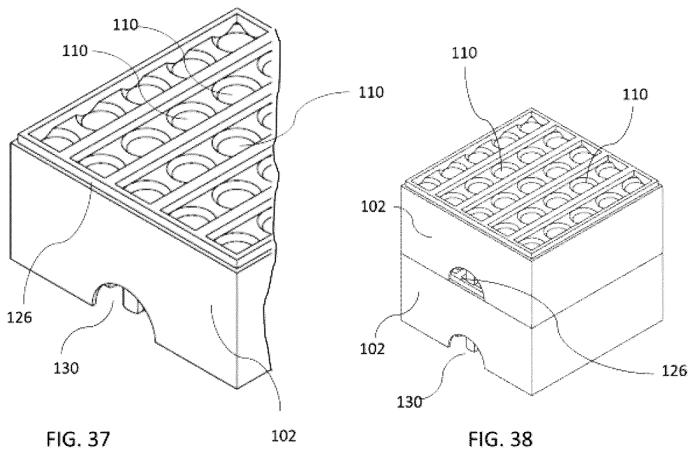

FIGS. 37 and 38 illustrate other features of the medical tray apparatus 100 in accordance with some embodiments of the present disclosure. The base tray 102 may include a peripheral lip 126 extending about at least a portion or all of the periphery of the base tray 12. The peripheral lip 126 is received within a correspondingly dimensioned recess 128 (FIGS. 33 and 34) on the underside of a second base tray 102 to enable multiple base trays 102 to be assembled, and transported or stored, in stacked relation as depicted in FIG. 38. In some embodiments, the base tray 102 may include a hand cutout 130, for example, a pair of cutouts 130 in opposed outer walls of the base tray 102 to facilitate engagement and lifting/carrying by the user or clinician.

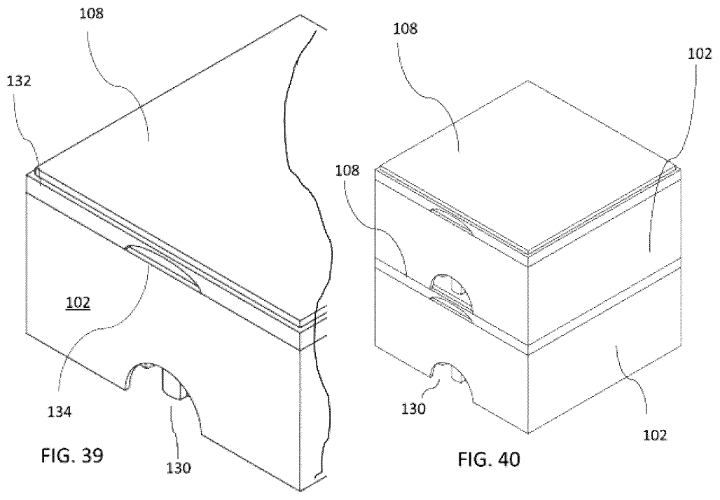

FIGS. 39 and 40 illustrate additional features of the medical tray apparatus 100 in accordance with some embodiments of the present disclosure. The cover 108 also may include a peripheral recess 132 extending about at least a portion its periphery. The peripheral recess 132 enables the cover 108 to be received within the recess 128 (FIGS. 33 and 34) on the underside of a second base tray 102 to enable multiple medical tray apparatuses 100 including the covers 108 to be arranged in stacked relation. The cover 108 also may include one or cutouts 134, for example, diametrically opposed cutouts 134 to facilitate engagement by the user or clinician. The cover 108 may be insulated and/or may contain a cooling medium therein.

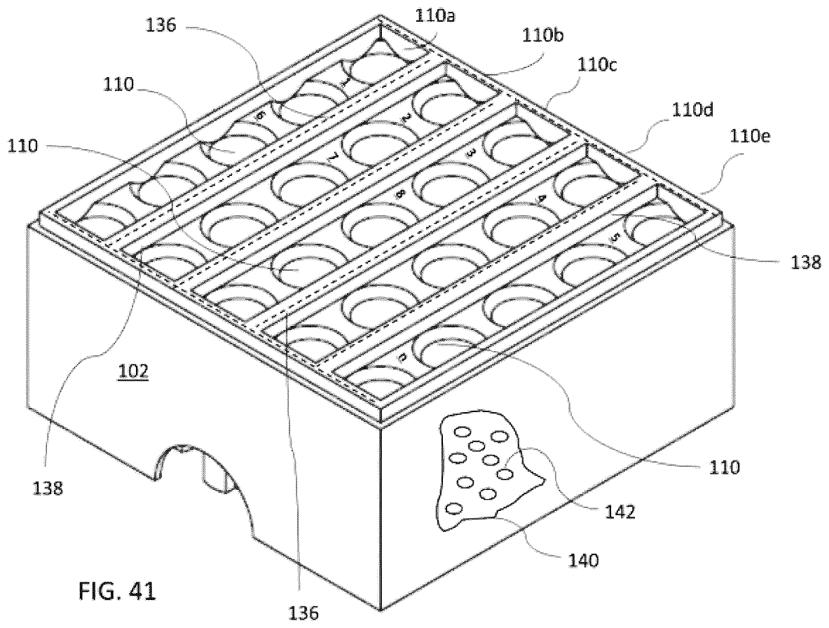

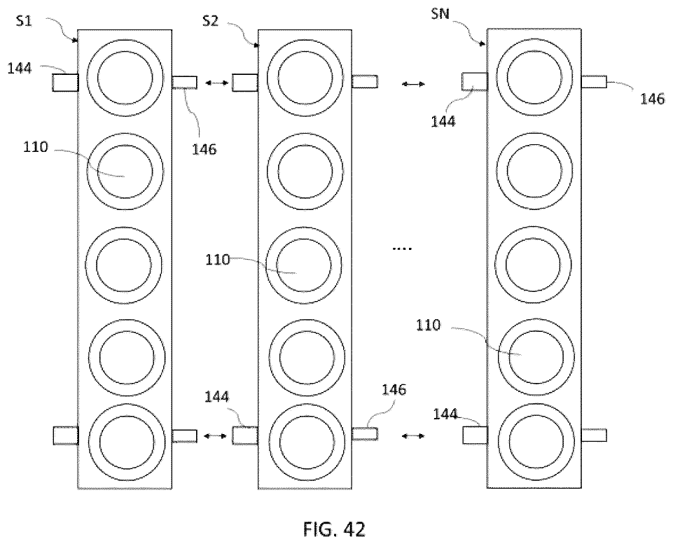

FIG. 41 depicts one or more illustrative embodiments of the medical tray apparatus 100 of the present disclosure. In certain embodiments, the base tray 102 may include indicia such as one or more alpha-numeric symbols (e.g., numbers, including numbers 1-n) indicating the number of individual cartridges used and/or remaining in the base tray 102. This feature facilitates inventory control and/or the monitoring of the dosing administered to the user. In other illustrative embodiments, the base tray 102 may be scored or include perforated lines 136 in the tray walls 138 separating the individual rows 110a-110e of the cartridge receptacles 110, and also along the periphery of the base tray 102. These features permit removal of one or more select rows 110a-110e of the individual cartridge receptacles 110 along with, in some embodiments, the accompanying individual cartridges 104. Thus, it is envisioned that a given row 110a-110e could correspond to a strip of a select dosing regimen. For example, a row of seven individual cartridge receptacles 110 and individual cartridges 104 may correspond to a seven day regimen of delivering, for example, antibiotics on a daily basis to a user.

In other embodiments, a first medical tray apparatus 100 may be arranged to contain individual cartridges 14 having a first fluid agent and one or more additional medical tray apparatuses 100 may contain individual cartridges 104 having one or more additional fluid agents. Each medical tray apparatus 100 may be color coded to correspond to the fluid agent contained within its respective individual cartridges 104. Other indicators other than color-coding may be utilized including, without limitation, particular markings, hashing, etc. disposed on the walls so the base tray 102. In some embodiments, each medical tray apparatus 100 may include one or more rows 110a-110e of cartridge receptacles 110 which can be separated along the perforated lines 136 in the manner described in connection with FIG. 41. In certain embodiments, a row from each medical tray apparatus 100 may be coupled to a row of one or more other medical tray apparatuses 100 to provide a treatment regimen for multiple diseases. For example, and without limitation, an immunization schedule for a child from birth to fifteen (15) months of age, in accordance with the protocol recommended by the Center for Disease Control (CDC), includes a vaccine regimen to address a number of medical conditions including hepatitis B, Rotavirus, diphtheria, haemophilus influenza type-b, pneumococcal bacteria, inactivated poliovirus, COVID-19, influenza, measles, mumps, etc. In illustrative embodiments, one medical tray apparatus 100 may be dedicated to contain the vaccine or fluid agent for each of the aforementioned diseases. The medical tray apparatus 100 may be color-coded to identify the specific disease. For example, the Rotavirus vaccine may be within a red-colored base tray 102 and the measles vaccine may be within a blue colored base tray 102. In certain embodiments, one cartridge row 110a-110e of cartridge receptacles 110 of each base tray 102 with the select vaccine may be separated as a first strip and coupled to one or more strips or cartridge rows 110a-110e of other base trays 102 having different vaccines. Any means to connect the cartridge rows 110a-110e from the multiple base trays 102 are envisioned including various mechanical couplings such as clips, a snap fit, tolerance fit, bayonet coupling, etc. Thermally or electrically activated couplings are also envisioned. The connected cartridge rows 110a-110e of the multiple base trays 102 may provide a comprehensive system containing the vaccines required to immunize a child from birth to fifteen months as per CDC protocol.

FIG. 41 also illustrates in a cut away section 140 within one or more walls of the base tray 102 depicting a reservoir, for example, within multiple layers of the walls of the base tray 102, containing the cooling medium 142.

FIG. 42 illustrates multiple strips of base trays 102 from different medical tray apparatuses 100 in the process of being coupled. Strip S1 corresponding to a row of cartridge receptacles 110 of a first medical tray apparatus 100 is shown being coupled to strip S2 corresponding to a row of cartridge receptacles 110 of a second medical tray apparatus 100. Multiple additional strips SN may be coupled in sequence to strip S2. The coupling mechanism may include multiple tabs and receptacles 144, 146 extending from the sides of the strip S1 . . . Sn. The multiple tabs and receptacles 144, 146 may be configured to engage in secured relation therewith through a clipping action, snap fit, tolerance fit etc. to form a combined unit of vaccines in association with a vaccine protocol.

In some other embodiments, the base tray 102 may be color-coded to the plunger of a syringe system, such as a BFS cartridge syringe system, to help minimize administration errors. For example, a BFS cartridges stored in cartridge receptacles 110 of a yellow base tray 102 could be removed and administered using a yellow plunger for a specific fluid agent, BFS cartridges stored in cartridge receptacles 110 of a blue base tray 102 may be removed and administered using a blue plunger for a second fluid agent, etc. Further, in some embodiments the syringe component of the delivery system may also be color-coded (e.g., a safety tag, or other component of a syringe may be color coded to the plunger to be used with the syringe).

In illustrative embodiments, the medical tray apparatus 100 may be fabricated from excess resin material produced, for example, as a byproduct or flashing, in the manufacture of the aforedescribed BFS cartridges and/or material discarded during use of the BFS cartridges.

### Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

Devices that are in communication with each other need not be in continuous communication with each other, unless expressly specified otherwise.

A description of an embodiment with several components or features does not imply that all or even any of such components and/or features are required. On the contrary, a variety of optional components are described to illustrate the wide variety of possible embodiments of the present invention. Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present invention provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present invention herein without departing from the scope thereof. Therefore, the above description should not be construed as limiting the invention, but merely as embodiments thereof. Those skilled in the art will envision other modifications within the scope of the invention as defined by the claims appended hereto.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A syringe assembly, which comprises:
a syringe housing:
a compressible cartridge at least partially positionable within the syringe housing, and having a fluid agent, the compressible cartridge including a compressible bellows segment;
a plunger configured for movement within the syringe housing to cause transition of the compressible cartridge from an initial condition to an at least partially compressed condition to compress at least the compressible bellows segment to dispense the fluid agent; and
a delivery member mountable to the syringe housing and configured for penetrating the compressible cartridge during transition of the compressible cartridge to the at least partially compressed condition into a subject.

2. The syringe assembly according to claim 1 wherein the compressible bellows segment of the compressible cartridge is configured to cooperatively engage internal structure of the syringe housing upon transition to the at least partially compressed condition thereby preventing removal of the compressible cartridge from the syringe housing and impeding transition toward the initial condition of the compressible cartridge.

3. The syringe assembly according to claim 2 wherein the compressible bellows segment of the compressible cartridge is configured to expand during transition to the at least partially compressed condition of the compressible cartridge.

4. The syringe assembly according to claim 3 wherein the syringe housing includes an internal annular rib segment, the internal annular rib segment being the internal structure of the syringe housing.

5. The syringe assembly according to claim 4 wherein the internal annular rib segment extends along a major portion of an inner dimension of the syringe housing.

6. The syringe assembly according to claim 4 wherein the internal annular rib segment is configured to engage one or more folds of the compressible bellows segment when in the at least partially compressed condition of the compressible cartridge.

7. The syringe assembly according to claim 6 wherein the plunger comprises a grasper component configured to engage the compressible cartridge to secure the compressible cartridge relative to the plunger.

8. The syringe assembly according to claim 7 wherein the grasper component of the plunger includes multiple grasping legs configured to deflect outwardly to engage the compressible cartridge.

9. The syringe assembly according to claim 8 wherein the multiple grasping legs of the grasper component are configured to engage a proximal collar of the compressible cartridge.

10. The syringe assembly according to claim 9 wherein the grasper component is configured to cooperate with the proximal collar of the compressible cartridge wherein a force required to release the grasper component from the proximal collar is less than a force required to release the one or more folds of the compressible bellows segment from the internal annular rib segment of the syringe housing such that the grasper component releases the plunger in response to retracting movement of the plunger when in the at least partially compressed condition of the compressible cartridge.

11. The syringe assembly according to claim 9 wherein the compressible cartridge includes a distal collar and wherein the syringe housing includes an internal annular shelf segment, the internal annular shelf segment cooperating with the distal collar of the compressible cartridge to secure the compressible cartridge within the syringe housing when in the initial condition of the compressible cartridge.

12. The syringe assembly according to claim 11 wherein the grasper component is configured to cooperate with the proximal collar of the compressible cartridge wherein a force required to release the grasper component from the proximal collar is less than a force required to release the distal collar from the internal annular shelf segment of the syringe housing such that the grasper component releases the plunger in response to retracting movement of the plunger when in the initial condition of the compressible cartridge.

13. The syringe assembly according to claim 1 wherein the plunger comprises an anti-activating component having a first pre-activation condition preventing movement of the plunger and dispensing of the fluid agent and a second activated condition enabling movement of the plunger and dispensing of the fluid agent.

14. The syringe assembly according to claim 13 wherein the anti-activating component includes a flag segment depending outwardly relative to an axis of the plunger when in the first pre-activation condition and movable to the second activated condition to enable movement of the plunger.

15. The syringe assembly according to claim 14 wherein the flag segment is detachable relative to the plunger to assume the second activated condition.

16. The syringe assembly according to claim 13 wherein the anti-activating component includes one or more deflectable arms configured to move from a radial outward condition corresponding to the first pre-activation condition and a radial inward condition corresponding to the second activated condition.

17. The syringe assembly according to claim 16 wherein the anti-activating component includes multiple deflectable arms.

18. The syringe assembly according to claim 16 wherein the one or more deflectable arms are configured to be received within the syringe housing to enable movement of the plunger when in the radial inward condition.

19. The syringe assembly according to claim 1 wherein the compressible cartridge includes visual indicia to facilitate identification of one of the fluid agent or type of compressible cartridge.

20. The syringe assembly according to claim 1 wherein the delivery member defines a subject end configured for application to a subject and an opposed cartridge end configured for penetrating the compressible cartridge.

21. The syringe assembly according to claim 20 wherein the cartridge end of the delivery member includes piercing structure configured for piercing the compressible cartridge.

22. The syringe assembly according to claim 20 wherein the delivery member comprises a cannulated needle.
